(19) United States
(12) Plant Patent Application Publication
(10) Pub. No.: US 2022/0338403 P1
(43) Pub. Date: Oct. 20, 2022

(54) X Mangave plant named 'Night Owl'

(50) ;

(71) Applicant: Hans A. Hansen, Zeeland, MI (US)

(72) Inventor: Hans A. Hansen, Zeeland, MI (US)

(73) Assignee: Walters Gardens Inc, Zeeland, MI (US)

(21) Appl. No.: 17/300,222

(22) Filed: Apr. 16, 2021

(51) Int. Cl. A01H 6/32

(52) U.S. Cl. PLT/373

(57) ABSTRACT

A new and unique X Mangave plant named ‘Night Owl’ characterized by compact habit of lanceolate foliage developing dark grayed-purplish color in strong ultraviolet light exposure. Leaves have large, widely-spaced, firm, marginal teeth and small, semi-flexible apical mucro. The new plant has numerous chartreuse flowers with burgundy blushing on tall, stiff, stoutly-branched scapes. The new plant is suitable as a potted houseplant plant, as a container plant for the patio or garden and for the garden or planted in the landscape, and the flowers are loaded with nectar for attracting hummingbirds.

Botanical classification: Manfreda x Agave hybrid.

Variety denomination: ‘Night Owl’.

### STATEMENT REGARDING PRIOR DISCLOSURES UNDER 37 CFR 1.77(b)(6)

The first non-enabling public disclosure of x Mangave ‘Night Owl’ was on Mar. 2, 2020 as a brief description and photo on two websites operated by Walters Gardens, Inc. Information for this website and plants for this sale were obtained from the inventor. The first sales disclosure of the new plant was on Jul. 20, 2020. No plants of x Mangave ‘Night Owl’ have been sold, in this country or anywhere in the world, nor has any disclosure of the new plant been made, more than one year prior the filing date of this application, and such disclosure within one year was either derived directly or indirectly from the inventor.

### BACKGROUND OF THE INVENTION

The present invention relates to the new and distinct X Mangave plant, X Mangave ‘Night Owl’ hybridized on Nov. 20, 2015 by the inventor at a wholesale perennial nursery in Zeeland, Mich., USA as a cross between ‘Silver Fox’ U.S. Plant Pat. No. 29,642 as the female or seed parent and ‘Inkblot’ U.S. Plant Pat. No. 29,431 as the male or pollen parent. Through trials at the same nursery the plant was referred to by the code 16-17-4. The new plant has been successfully asexually propagated initially in 2018 by shoot tip tissue culture at the same nursery in Zeeland, Mich. This asexual propagation system has been found to produce stable and identical plants that maintain all the unique characteristics of the original plant in successive generations.

### BRIEF SUMMARY OF THE INVENTION

X Mangave ‘Night Owl’ differs from its parents as well as all other Manfreda, Agave and x Mangave known to the applicant. The most similar x Mangave cultivars known to the inventor are: ‘Mayan Queen’ U.S. Plant Pat. No. 31,312, ‘Catch a Wave’ U.S. Plant Pat. No. 29,723, ‘Mission to Mars’ U.S. Plant Pat. No. 29,393, ‘Desert Dragon’ U.S. Plant Pat. No. 31,311, ‘Red Wing’ U.S. Plant Pat. No. 30,650, ‘Freckles and Speckles U.S. Plant Pat. No. 30,904 and ‘Black Magic’ copending U.S. Plant Patent Application.

‘Mayan Queen’ has a more upright, larger, more arching habit with foliage that is narrower and lighter silver with purple spotting. ‘Catch a Wave’ has a larger habit, leaves that are a more silvery-blue coloration and the leaves are more folded and sinuate. ‘Mission to Mars’ has a much larger habit and the foliage is more reddish colored, and lacks the large marginal teeth. ‘Desert Dragon’ has a more horizontal habit with narrower leaves with a more undulating margin and lacks the large marginal teeth. ‘Red Wing’ has more numerous leaves and the coloration is more reddish foliage and the marginal teeth are smaller and more evenly spaced. ‘Freckles and Speckles’ has narrower, shorter foliage with smaller spotting, lacks the large marginal teeth, and has a smaller, less arching habit. ‘Black Magic’ has similar dark foliage, with narrower shape with less undulation, flatter more arching habit, and the teeth are smaller and more evenly spaced.

‘Silver Fox’ has a more compact habit and lacks the grey-purple spotting. ‘Inkblot’ has flatter growth habit, the leaves arch more, are longer, thinner and narrower, with less burgundy airbrushing, and the marginal teeth are smaller.

The new plant, ‘Night Owl’, is unique from all of these Agave, X Mangave and Manfreda known to the inventor by the following combined traits:
1. Low mounded habit with 30 to 40 leaves at flowering;
2. Foliage is undulated with widely-spaced, large, firm, marginal teeth and small, semi-flexible apical mucro;
3. Leaves develop dark grayed-purplish overcast with strong ultraviolet light exposure;
4. Moderate growth rate;
5. Numerous flowers of chartreuse with burgundy blushing on stiff, tall, stoutly-branched scapes.
6. The foliage is upright with the leaf tips arching.

### BRIEF DESCRIPTION OF THE DRAWINGS

The photograph of the new plant demonstrates the overall appearance of the new plant including the unique traits as a three-year-old plant grown in a full-sun trial garden with supplemental water and fertilizer as needed. The colors are as accurate as reasonably possible with color reproductions. Ambient light spectrum, temperature, source and direction may cause the appearance of minor variation in color.

FIG. 1 shows the habit of the new plant from above.

FIG. 2 shows a close-up of the foliage and marginal teeth.

FIG. 3 shows a close-up of the flowers and upper scape.

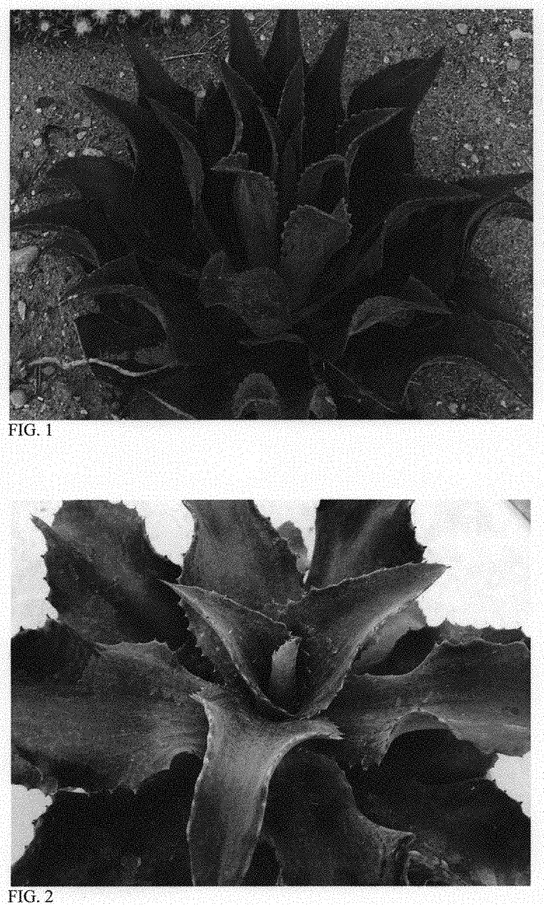

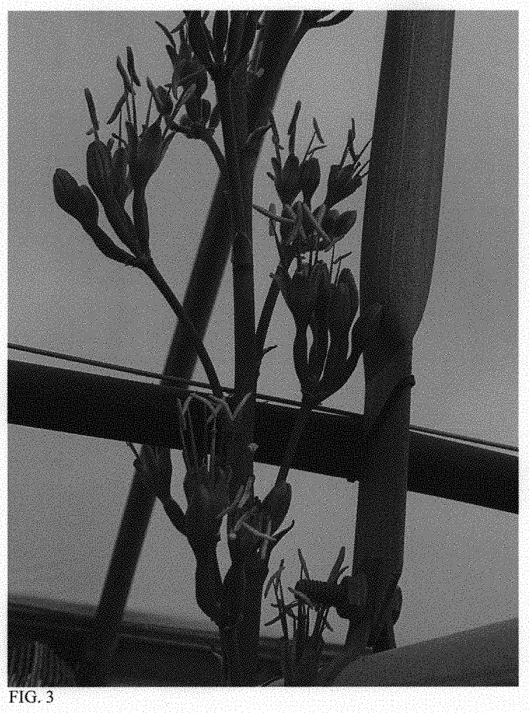

### DETAILED BOTANICAL DESCRIPTION

The following descriptions and color references are based on the 2015 edition of The Royal Horticultural Society Colour Chart except where common dictionary terms are used. The new plant, X Mangave ‘Night Owl’, has not been observed under all possible environments. The phenotype may vary slightly with different environmental conditions, such as temperature, light, fertility, moisture and maturity levels, but without any change in the genotype. The following observations and size descriptions are of two and three-year old plants in a commercial wholesale greenhouse and in a full-sun display garden in Zeeland, Michigan with supplemental water and fertilizer as needed.
Botanical classification: x Mangave hybrid (Manfreda x Agave);
Parentage: ‘Silver Fox’ as the female or seed parent and ‘Inkblot’ as the male or pollen parent;
Propagation: Crown offsets and sterile plant tissue culture;
Time to initiate roots from tissue culture: About 21 days;
Growth rate: Moderate;
Crop time: About 16 to 20 weeks to finish during lengthening spring days from an established 25 mm tissue culture plug to a 65 mm diameter container;
Rooting habit: Fleshy, lightly branching, with roots up to 30 cm long;
Root color: Nearest RHS 158C;
Plant shape and habit: Succulent herbaceous perennial with basal rosettes of up to about 34 fleshy leaves radially emerging and arching from central rhizome, producing a symmetrical, low, rounded mound;
Plant size: Foliage height about 25.0 cm tall from soil line to the top of the leaves and 45.0 cm wide at soil level, flowering to about 252.0 cm tall and to about 18.0 cm wide at the widest flowers;
Foliage description: Lanceolate; simple; sarcous; glabrous; fibrous; slightly asperous and glaucous adaxial and abaxial; margins coarsely dentate with firm teeth; apex acute with short semi-flexible mucro; base truncate, sessile, clasping; concavo-convex and slightly sinuate with maturity;
Leaf size: To about 23.0 cm long, about 7.0 cm wide about 8 cm from base and constricting near base to about to about 6.5 cm wide, 1.8 cm thick and 2.0 cm deep; average about 21.5 cm long, 6.5 cm wide about one-third distance from base; usually bi-laterally symmetrical;
Marginal teeth: About 3.0 mm long and to about 10.0 mm apart in distal on-third, average about 7.6 mm apart;
Foliage fragrance: None observed;
Leaf blade color:
   Adaxial (lower ultraviolet exposure).—Young between RHS 188C and RHS N187C with speckles nearest RHS N189B and mature nearest RHS 137B with spotting nearest RHS N187B.
   Abaxial (lower ultraviolet exposure).—Young between RHS 188C and RHS N187C without significant spotting and mature nearest RHS 137C without significant spotting.
   Adaxial (lower ultraviolet exposure).—Young and mature between RHS N187B and RHS N187A having spots nearly completely overlapping.
   Abaxial (lower ultraviolet exposure).—Young nearest RHS N187D with undertones of nearest RHS N187A and mature nearest RHS N138A with spotting of nearest RHS N187A.
   Marginal teeth.—Young adaxial and abaxial 3.0 mm tips; color in lower ultraviolet light nearest RHS N163A and on older leaves adaxial and abaxial tips between RHS 166A and RHS 166B, and on high ultraviolet light exposure adaxial and abaxial tips nearest RHS N186A.
Mucro: Semi-flexible; sharp; straight, about 7.0 mm long and 1.0 mm across;
Mucro color: Nearest RHS 200A;
Petiole: Sessile;
Veins: Parallel; not distinct;
Peduncle: Terete; glaucous; glabrous; stiff; strong; upwardly; heavily branched with about 23 short branches; about 252.0 cm long and 2.4 cm diameter at base and 1.8 cm diameter below first branches; branches to about 8.0 cm long and about 0.4 cm diameter at base; branch aspect upwardly and outwardly;
Peduncle color: Proximally nearest RHS N187A, distally nearest RHS 146B with blush nearest RHS N186C ; branches nearest RHS N187A in proximal region and nearest RHS 146B distally with blushing of nearest RHS N187A reduced distally to only nearest RHS 146B;
Pedicel: Terete; glaucous; glabrous; stiff; strong; average about 15 mm long and 3.0 mm diameter;
Pedicel color: Nearest RHS 146D;
Cauline bracts: About 90 per scape below flowers and one at each branch; lanceolate; simple; sarcous, glabrous; margins finely dentate with flexible teeth to about 0.5 mm long; apex narrowly acute with semi-flexible mucro to about 4 mm long; base truncate, sessile, clasping; typically adpressed to peduncle in proximal one-half with distal one-half divergent and slightly twisted around peduncle; to about 10.0 cm long and about 2.0 cm wide near base, decreasing distally;
Cauline bract color: Adaxial nearest RHS 137B with light blush nearest RHS N187A in the center and a strong blush of between RHS N187A and RHS 187B on the margins; abaxial between RHS 189A and RHS N138A with a light to moderate blush of between RHS N187A and RHS 187B in the centers and the margins a blend between RHS N187A and RHS 187B;
Buds one day prior to opening: Elongated globose; about 4.5 cm long and about 9.0 mm across at bulb, base about 4.0 mm diameter at ovary and 3.0 mm diameter between bulb and ovary, bulb about 2.5 cm long;
Bud color: Between RHS 143A and RHS 146D in ovary portion, basal tepal portion between RHS 143D and RHS 146D and distal one-half nearest RHS 146D and lightly maculate with nearest RHS 183B;
Flower description: Perfect; incomplete; actinomorphic; about 8.0 cm long to exserted anthers and opening to about 3.0 cm across at outside of anthers; corolla to about 4.7 cm long and 2.6 cm wide at apex; lasting about 3 to 4 days per flower; flowering period about three weeks once plant is mature, approximately three-years-old; producing abundant nectar; (only observed in a partly shaded greenhouse in the winter season);
Inflorescence: Approximately 85 to 100 flowers per scape; aspect mostly upright; flowering portion about 62.0 cm tall and about 18.0 cm across;
Flower fragrance: Faintly sweet;
Tepals: Six in two sets of three; glabrous and slightly lustrous adaxial and abaxial; acute apex and fused base; outer set about 27.0 mm long, fused in basal 10.0 mm and about 8.0 mm wide near middle; inner set about 27.0 mm long and 7.0 mm wide near middle; outer set creased on abaxial side along margin where overlapped with outer set while in bud;
Tepal color: Outer tepal set adaxial proximally between RHS 145C and RHS 146D, distally nearest RHS 162B and moderately maculate with nearest RHS 187B, abaxial between RHS 145C and RHS 150C with heavy blush distally of nearest RHS 187B; inner tepal set adaxial proximally between RHS 145C and RHS 150C, distally with slight blush of nearest RHS 187B and a thin margin about 0.5 mm wide of nearest RHS 187A, abaxial center between RHS 145C and RHS 150C with a margin about 2.0 mm wide nearest RHS 162C and moderately maculate with nearest RHS 187B;
Androecium: Six;
   Filaments.—Six; straight; glabrous, lustrous; terete distally, slightly applanate and adnate to inner corolla in proximal 5.0 mm; about 40.0 mm long and 2.0 mm across at base; color nearest RHS 162D and moderately maculate with nearest RHS 183B.
   Anther.—Dorsifixed; longitudinal; straight, oblong; about 18.0 mm long and 2.5 mm diameter before opening; color adaxial nearest RHS 151D, abaxial nearest RHS 151D and moderately maculate with nearest RHS 187A.
   Pollen.—Abundant; color nearest RHS 14A.
Gynoecium: Single; 64.0 mm long;
   Style.—44.0 mm long and 2.0 mm diameter at base; color at base nearest RHS 162D moderately maculate with nearest RHS 183B.
   Stigma.—Turbinate, apex tri-lobed; distally puberulent; about 2.0 mm tall and 3.0 mm across top; color side nearest RHS 156C.
   Ovary.—Inferior; oblong; about 18.0 mm long and 5.5 mm across; surface smooth; lustrous; with three distinct raised ridged running along middle of outer tepals to base of ovary; color between RHS 143A and RHS 146D.
Fruit: Not yet observed;
Seed: Not yet observed;
Disease resistance: x Mangave ‘Night Owl’ has not been observed to be resistant to diseases common to other x Mangave beyond that which is normal for Agave or Manfreda. The plant is xeromorphic and survives well with minimal water once established. Hardiness at least from USDA zone 9 to 11. Full extent of winter hardiness has not been tested.

I claim:

1. A new and distinct cultivar of ornamental X Mangave plant named ‘Night Owl’ as herein described and illustrated.
